(19) United States
(12) Patent Application Publication
(10) Pub. No.: US 2004/0176666 A1
(43) Pub. Date: Sep. 9, 2004

(54) Health and wellbeing monitoring and advisory system

(76) Inventor: Daryl Chait, Victoria (AU)

Correspondence Address: NEEDLE & ROSENBERG, P.C., SUITE 1000, 999 PEACHTREE STREET, ATLANTA, GA, 30309-3915, US

(21) Appl. No.: 10/483,936

(22) PCT Filed: Jul. 17, 2002
(86) PCT No.: PCT/AU02/00956

(30) Foreign Application Priority Data
Jul. 20, 2001 (AU) PR 6515

(51) Int. Cl. A61B 5/00; G06F 17/00

(52) U.S. Cl. 600/300; 128/921; 128/903; 128/904; 235/375; 708/132

(57) ABSTRACT

A health and wellbeing system is disclosed which has particular application to monitoring dietary intake. The system includes a user mobile telephone (15) which a user uses to transmit information relating to food products to be consumed by a user or other health related issues to a database (12) and processor (14) at a remote location. The database (12) maintains data relating to the user, and which is personal to the user, as well as general data which relates to dietary content of food products and other health and wellbeing parameters. Upon transmission of information from the user mobile telephone (15) by SMS message to the database (12), the processing section (14) determines an appropriate response based on the specific user data stored in the database section (12a) and/or general information stored in the database section (12b), and transmits a message back to the user by way of SMS message to the telephone (15). The information supplied may relate to advice as to whether the user should consume a food product, undertake a specific activity or may contain general information as to the user's progress with a health and wellbeing program.

### FIELD OF THE INVENTION

This invention relates to a health and wellbeing monitoring and advisory system and, in particular, to a diet monitoring system for enabling a user to monitor one or more characteristics of food consumed by the user and for alerting a user when intake of that characteristic in a given time period exceeds or approaches a pre-determined level.

### BACKGROUND ART

Apparatus for monitoring a person's diet generally comprise calorie counters in which a person can input into a counter the number of calories in a product to be consumed by the user. This generally requires the user to be able to access the number of calories the food product has from the container of the food product and key that data into the device. The device maintains a count of the amount of calories consumed so that at any given time the user has an indication of the number of calories consumed.

These devices have a number of limitations and, in particular, the limitation of the user having to ascertain the number of calories in a particular product before relevant data can be inputted into the counter.

There is also a need for a more general health and wellbeing monitoring and advisory system which can provide rapid monitoring and advice of a wide range of health and lifestyle issues which may or may not include advice relating to dietary intake. The monitoring of other health related parameters and advice back to a user relating to those parameters can also assist in maintaining a healthy lifestyle.

### SUMMARY OF THE INVENTION

The present invention seeks to provide a more sophisticated health and wellbeing monitoring system and also a diet monitoring system which enables a consumer to monitor the intake of food characteristics.

The invention, in a first aspect, may be said to reside in a health and wellbeing advisory system, including:

a central database which holds information and data relating to a specific user, and information relating to health and wellbeing parameters, the central database being contactable by a transmitter used by a user, which transmitter is able to transmit information relating to a user to the database;

processing means for receiving the user information and determining a response to the user information based on the data relating to the specific user and/or the information relating to the health and wellbeing parameters; and

return transmitting means for transmitting a response back to the user's transmitter.

Thus, the invention enables a user to transmit information relating to the user which may be information relating to food the user intends to consume, an activity the user intends to perform, or information relating to user wellbeing characteristics, such as sleep patterns, stress, or the like. The database and processing means is then able to receive that information and from the user information which is retained in the database and/or additional wellbeing parameters maintained in the database, the system is able to formulate a response possibly in accordance with certain protocols for transmission back to the user to provide the user with advice relating to the situation of interest to the user. For example, the information may be dietary information which relates to a food item the user is about to consume.

The information transmitted may be nutritional information relating to that food product, including calories, fat content, fibre content, alcohol content, etc., and the processing means is able to receive that information and determine from the user information whether the user should eat that food product. One example of how the processing means may determine the response is to first scrutinise the user information in the database which may indicate that the user is diabetic, and then compare the food items which the user wishes to consume with information in the health and wellbeing parameters, and may therefore decide whether the food is suitable for the user to consume and transmit a message accordingly. The information may also simply relate to the calorie content and may compare the calorie intake of the user if the user consumes that item with calories already consume by the user during the course of a predetermined period such as that day, and may advise the user that if the item is consumed, the user will have consumed food products above the recommended calorie intake, etc. in a given period. The information may also relate to sleep patterns or stress and may provide information back to the user indicating that the amount of sleep the user is getting over a predetermined period is not sufficient, or that the stress the user has experienced over a certain period is excessive. This enables the user to take note of these parameters, which may otherwise slip the user's attention and the user is then in a position to take remedial action or seek medical advice.

Preferably the processing means is stored with predetermined protocols against which the response is determined based on those protocols, as well as the user information transmitted to the central database, and the information and data relating to the specific user and/or the health and wellbeing parameters.

Preferably the transmitter is a wireless transmitter.

Preferably the wireless transmitter is a mobile telephone and the database receives information identifying the user by the mobile telephone number which is transmitted with the data to the database, and the return transmitting means is a mobile telephone transmitting means which transmits the response to a user by way of a mobile telephone call.

In one embodiment, the information is transmitted from the user mobile telephone by an SMS message, and the response is transmitted back to the user from the return transmitting means by an SMS message.

Preferably the database includes a first database section which includes personal records at which specific user information is stored relating to the specific users of the system, and a second database section in which health and wellbeing parameters are stored.

Preferably the database includes an SMS modem for receiving the information from the user and for supplying the information to the database and the processing means.

Preferably a health and nutrition evaluation centre is coupled to the system so that information relating to a specific user can be input into the system by consultation with a lifestyle consultant.

Preferably the health and nutrition evaluation centre is connectable to the system over the Internet.

Preferably the user transmitter includes input means for inputting data into the system for transmission to the database.

In one embodiment, the input means comprises a scanner connectable to the wireless transmitter so that a bar code of a product can be scanned to provide the information relating to the product.

In another embodiment, the transmitter includes a menu of products and the input is by selection from a draw down menu in the transmitter.

In a still further embodiment, the transmitter includes voice recognition software and the input is by way of a spoken word which is recognised by the software in the transmitter, so that data relating to the voice input is transmitted by the transmitter to the database.

Preferably the user transmitter includes a display for displaying the quantity of the food characteristic consumed by a user.

Preferably the apparatus also include alarm means for generating an alarm when the total amount of a particular food characteristic consumed by a user approaches or reaches a pre-determined level. This feature therefore enables a person to become aware when the person's calorie intake, alcohol intake, fat intake or the like approaches or reaches a pre-determined level so the person can be warned they have consumed their daily allowable quantity of, for example, calories, or is approaching a prescribed intake level.

Preferably the user information stored in the central database includes the user's height, weight, age and the like, specific activities the user performs such as sporting activities and the like, as well as intended objectives which the user wishes to achieve, such as weight loss, muscle building, etc., and general health information.

Preferably the processing means also includes processing means for calculating an ideal intake of calories having regard to the user information and data and the requirements or end result the user wishes to achieve. This enables an ideal intake of calories, fat, and other parameters of food products, to be calculated dependent on the user's particular circumstances and what the user does actually wish to achieve so that those calculations can be used as a basis for determining whether the consumption by the user is approaching or about to exceed a predetermined limit.

In one preferred embodiment of the invention the system also allows input of information relating to the calories burnt by a user during the user's daily activity. Such a system may include a heart monitoring device associated with the transmitter which monitors a user's heart so as to obtain an indication of the heart rate and therefore the amount of calories which would be burnt by the user in the day and this information can also be transmitter to the database for combination with counts of calories consumed to enable a count of the actual net intake of calories or net loss of calories during the course of a day to be calculated and used as a basis for determining whether intake is approaching or exceeding a predetermined limit, or for that matter, dropping below a predetermined limit.

In a further aspect, the invention may be said to reside in a diet monitoring system, including:

a central facility having:

(i) memory means for storing a databank of data relating to food products and characteristics of those food products;

(ii) receiving means for receiving a communication signal;

(iii) processing means for receiving the communication signal and processing the signal so that the characteristic of a food product to be consumed by a user can be determined from the memory means and stored to provide a measure of the consumption of the particular characteristic in a given time period;

user communicator means for communicating with the central facility to provide the communication signal, and having input means for input of information relating to a food product to be consumed; and

transmitter means for transmitting information relating to the food to be consumed back to the user.

This aspect of the present invention avoids the need of a user having to determine a particular characteristic of a food product such as number of calories, fat content, alcohol content, fibre content, etc., because that information can be stored in the memory means. Thus, when user inputs data relating to a food such as the name of the food, the processing means can simply extract the number of calories, fat content, fibre content, alcohol content, etc. relating to that food and maintain a count of the characteristic so that the accumulation of a particular food characteristic over the course of a period of time can be monitored.

In the preferred embodiment of the invention, the user communicator means includes indicating means for providing an indication as to the quantity of a food characteristic consumed by the user.

Preferably the indicating means comprises a display for displaying the quantity of total characteristic consumed by a user.

Preferably the communicator means also include alarm means for generating an alarm when the total amount of a particular food characteristic consumed by a user approaches or reaches a pre-determined level.

In a preferred embodiment of the invention the input means comprises a scanner so that a barcode on a food product can be scanned to input data relating to the food product such as calorie content, fat content, fibre content, alcohol content, etc.

The input means preferably further includes a keypad for manual input of data in the event that a food product does not include a barcode.

The invention in a further aspect may be said to reside in a diet monitoring system including:

a mobile telephone for transmitting information and receiving a response;

memory means for storing data relating to a plurality of food products and characteristics of those food products;

input means associated with the telephone for receiving data relating to a food to be consumed by a user and transmitting that data by a mobile telephone call;

processing means for comparing the data received by the input means with the data stored in the memory so as to extract from the memory an amount of at least one of the characteristics of the food the consumer is to consume, and transmitting the response to the mobile telephone; and

display means associated with the telephone for displaying that response.

This aspect of the invention has particular application in enabling a user to determine the amount of a particular characteristic of a food such as fat content, calorie content, alcohol level, etc. which a user is about to consume so that the user can make a judgement as to whether the user does wish to consume that product. This aspect of the invention has particular application in a restaurant environment to enable a user to, for example, scan a barcode alongside a menu item so that the particular characteristics of the menu item such as fat content, calorie content, alcohol content, fibre content, etc. can be extracted from the memory means and displayed so the user is appraised of the amount of that characteristic in the food the user intends to consume before the user decides on the menu item and consumes the menu item.

Preferably the system also includes alarm means for generating an alarm when the total amount of a particular food characteristic consumed by a user approaches or reaches a pre-determined level.

In a preferred embodiment of the invention the input means comprises a scanner so that a barcode on a food product can be scanned to input data relating to the food product such as calorie content, fat content, fibre content, alcohol content, etc.

The input means preferably further includes a keypad for manual input of data in the event that a food product does not include a barcode.

### BRIEF DESCRIPTION OF THE DRAWINGS

Preferred embodiments of the invention will be described, by way of example, with reference to the accompanying drawings in which:

FIG. 1 is a diagram of a system embodying the invention;

FIG. 2 is a block circuit diagram of the apparatus of FIG. 1;

FIG. 3 is a view of a mobile phone display according to one embodiment of the invention; and

FIG. 4 is a view of a menu with which one embodiment of the invention can be used.

### DESCRIPTION OF THE PREFERRED EMBODIMENTS

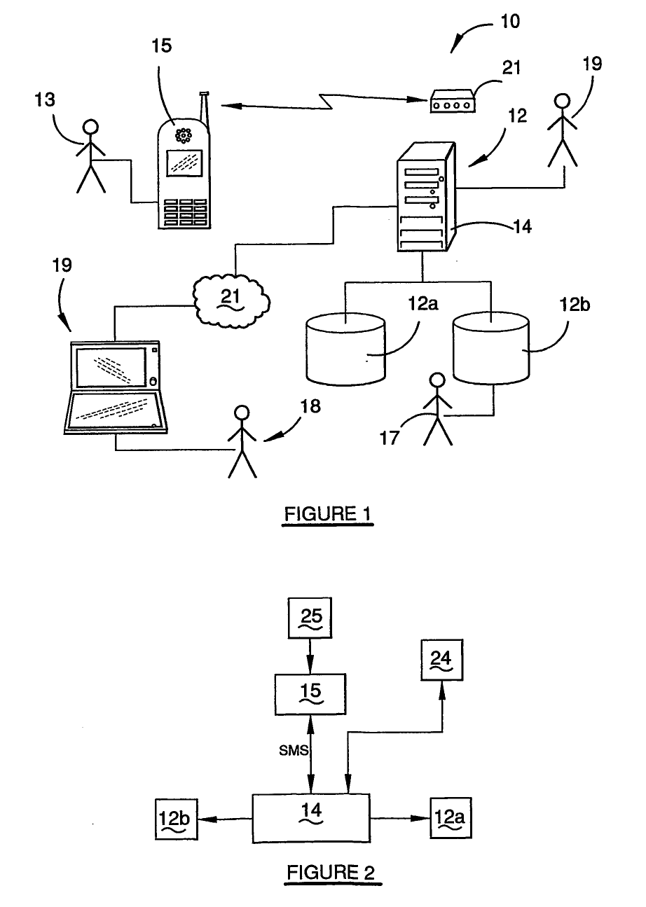

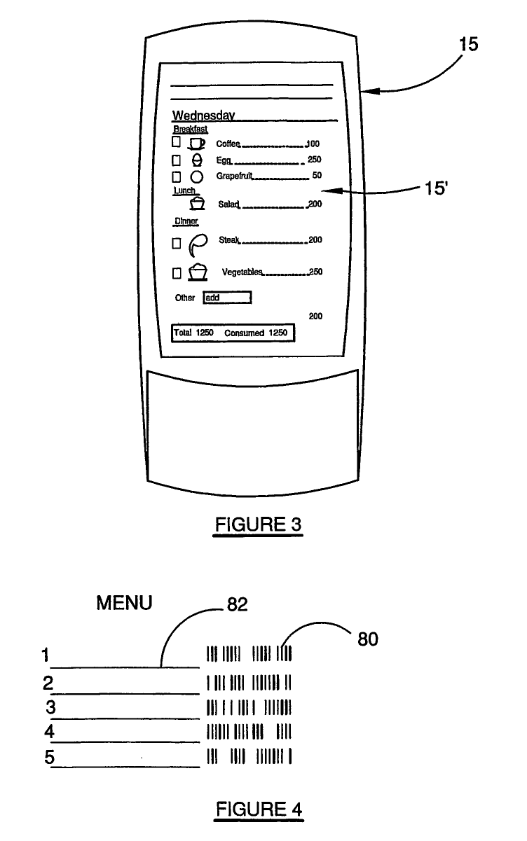

FIG. 1 shows a first embodiment of the invention in which a monitoring and advisory system 10 is shown.

With reference to FIG. 1, a system embodying the invention is described. The system 10 includes a central database 12 which includes a processor 14 for processing information and determining responses relating to information received, and also information maintained in the database 12.

The database 12 is basically divided into two parts. The first part 12a comprises personal records of all users of the system. The personal records include personal data relating to the user, such as age, height, weight, general physical wellbeing including specific health related issues. Such information may include specific information to ailments suffered by the user, such as being a diabetic, hearth problems, and then the other specific information which relates to the user and needs to be monitored or taken into account in relation to food consumption by the user, or other activity which the user performs.

The other part of the database 12 comprises part 12b which includes parameters relating to general health and wellbeing as well as nutritional information. The part 12b may include nutritional information relating to a vast range of food products, including calorie content, fat content, fibre content, alcohol content, and the like. This part of the database may also include specific information relating to various categories of people such as food products diabetics should not consume, food products or other activities which people with heart conditions should not eat or perform, good sleep patterns and stress management levels dependent on particular classes of people and activities which people perform, and other general health and wellbeing data.

A user 13 communicates with the database 12 by way of the user's mobile phone 15. This communication will be described in more detail hereinafter.

A user wishing to avail him or herself of this system will, in general, meet with a lifestyle consultant 17 at a health and nutrition and evaluation centre 19. Personal data relating to the user 13 is therefore obtained, including all of the information referred to above, and that information is forwarded to the database 12 via the Internet 21. A program can also be loaded into the user's mobile telephone 15 to record what the user eats and gives advice on health and nutrition. The user will enter into the phone details of what the user eats and what exercise the user does. The information can be entered by manually punching information into the keypad of the telephone, or bringing up a loaded menu and scrolling through the menu until the food item is identified. Such a menu as displayed on a mobile telephone is illustrated in FIG. 2. In a further embodiment, the input of data may be by way of voice recognition in which the mobile telephone is loaded with voice recognition software so that the nature of food consumed or other activity the user is to perform can simply be voiced into the telephone, recognised by the telephone and transmitted to data for transmission by the telephone.

The lifestyle consultant 17 at the centre 19 will also teach the user 13 how to use the mobile telephone in order to properly transmit data in accordance with the user's requirements, and to read messages which are transmitted back to the telephone 15 from the database 12.

The information which is loaded into the part 12b of the database is supplied by a nutritionist, medical professional and other health professionals 17.

The processing section 14 of the database 12 also requires decisions to be made based on information supplied by the user 13 via the mobile telephone 15, and the information contained in the database 12a relating to that user, and also the general information in the database 12b.

Information for controlling the processing section 14 is supplied by a medical professional 19 or other qualified person in which various protocols are loaded into the processing means for processing information supplied by the user 13 and the information in the databases 12a and 12b, so that a proper response can be supplied to the user.

The protocols are made by the processor based on various rules which are programmed into the system so that automated decisions can be made. However, consultants may be on hand to provide specific information into the system for transmission back to the user if the system decides that the protocols it has are not sufficient to provide an answer to the user. The protocols may relate to various classes of people, such as those who are diabetic, cholesterol sensitive, lactose intolerant, etc., with specific rules for deciding how to deal with food products such people intend to consume or other activities such people intend to undertake. In one specific embodiment, when a user inputs details of food products consumed, the processing section 14 can break down the food products consumed into basic nutritional elements by looking up the ingredients in the nutrition database 12b. Such a breakdown may include the following items: protein, carbohydrate, sugars, saturated fat, polyunsaturated fat, monosaturated fat, calcium, sodium, potassium, magnesium, bicarbonate, chloride, vitamin A, vitamin B, vitamin C, vitamin D and vitamin E. The intake of each ingredient is accumulated and compared against the user's daily allowance so that the message can be sent back, warning if the daily allowance is being exceeded or is about to be exceeded. Furthermore, the system may monitor the intake of these vitamins and other substances and issue a warning if the user's diet does not appear to have sufficient of the vitamins or other substances.

The information supplied by the user 13 is transmitted by the mobile telephone 15 as an SMS message to modem 21 connected to the database 12. That information is then supplied to the database 12 for processing by the processor 14, and a response is returned from the modem 21 by way of an SMS telephone call to the mobile telephone 15.

In one embodiment of the invention, the mobile telephone 15 can transmit information only once a day to the modem 21 and database 12. For example, all the calorie intake and other information entered by the user 13 into the mobile telephone 15 is simply stored and transmitted as one message. A single message may be received in response to that information, advising the user 13 of the user's progress, health intake, and the like, in relation to the objectives which the user wishes to obtain and which are stored in the part 12a of the database which relates to that user. This simply reduces the amount of communication from the user telephone 15 to the database 12. However, the user has the option of transmitting information at any time and seeking an immediate response in relation to a particular inquiry.

The obtaining of an immediate response may be essential in cases of serious health related issues, and whether or not the user requires immediate advise as to whether the user should undertake a particular activity or consume a particular food product. In such an instance, the user transmits the information immediately from the mobile telephone 15 by way of SMS message to the modem 21, and receives a response in respect of that query which is displayed on the display of the telephone 15 as an SMS message. The response may simply indicate that the user should not consume the food product intended or perform the activity or, alternatively, the user may perform the activity or consume the food product. The message may also include further information that, if the user does consume the food product, the user should exercise or undertake some other activity in combination with the consumption of that product. If the user is advised not to consume the food product, the processor 14 may suggest alternative food products which the user may wish to consider.

The information which the user transmits may also be in the nature of health related issues not being dietary issues. For example, sleep patterns and stress levels may be monitored by the system and the user may input sleep patterns and stress levels into the telephone for transmission to the database 12. The database 12 records that information against the user and therefore can monitor sleep patterns and stress levels and, if those levels depart from healthy levels, as maintained in the database 12b, a message can be transmitted to the user advising the user to take remedial action or seek medical guidance.

The degree to which personal information relating to the user can be input into the system depends on the requirements of the user and the user's general health and wellbeing. The information can be quite specific and go to the levels of having the user take blood tests and the like, so that a detailed medical history of the user is recorded in the user record in the database 12a.

In the preferred embodiment of the invention, the method of identifying the specific user is the user's mobile telephone number which is used to transmit information relating to the user to the database 12. That telephone number is used as a method of returning information to the user by way of SMS message to the user's mobile telephone.

FIG. 2 shows a block diagram of the database at processing section 12 of FIG. 1.

The processor 14 may include an additional memory 24 which serves as a short term memory for recording daily consumption or daily requirements of the user, which is just updated during the course of a day and then loaded into the user profile in the database 12a. The memory 24 can then be cleared on a daily basis for re-use in the next day. This enables an easy method of maintaining the daily count of intake by a user which can be compared to the user's daily required intake or the like, which is stored in the user's database 12a.

The database 12a is coupled to the processor 14 for storing data relating to the user of the device such as sex, height, weight, and other data relating to objectives the user wishes to maintain such as particular calorie intake, fat intake, or the like. Database 12b is also coupled to microprocessor 14 and stores the databank of food products and also characteristics of those food products such as fat content, calorie content, fibre content, alcohol content, etc.

In use, products to be consumed by a user will be provided with a bar code which includes an indication of the food product such as the name of the product and also data relating to the contents of the food product such as fat content, calorie content, fibre content, alcohol content (if applicable) and any other desired characteristic or parameter of the product. The barcode may also include data relating to the price of the product so that the conventional price data, etc. and the data relating to the characteristics of the food product mentioned above are contained within the one barcode. However, separate barcodes could be employed for this purpose if desired and appropriately coded so as to not interfere with the operation of one another.

When a user wishes to consume the food product, the user scans the barcode on the product with the a barcode scanner attached to telephone 15 so that the scanner passes over the barcode so that the data on the barcode is read by the scanner and supplied to the microprocessor 14. However, as mentioned previously in other embodiments, the manner of inputting data may be by way of input into the keys of the mobile telephone, or by way of voice recognition.

The preferred embodiment of the invention also enables the user to send an update of information relating to the user, for example, on a daily basis, which may include dietary information which has been stored in the telephone and which has been consumed by the user, exercise details, sleep patterns, stress levels and all other information which the user regards as applicable to his or her wellbeing. This information is received by the database 12 and processor 14, and the database 12 and processor 14 respond by SMS message to the mobile telephone 15 for a suitable reply and advice. The advice is generated based on the user's personal profile maintained in the database 12a and the general data retained in the database 12b. The processor 14 includes the set of rules and protocols which determine the advice which is provided by the processor 14 back to the mobile telephone 15. For most applications, a single transmission on a daily basis is sufficient to monitor and provide information relating to the user's health and wellbeing. However, people with specific medical conditions, and in need of data on an instantaneous basis, can transmit information relating to food products to be consumed and activity to be undertaken immediately to the database 12 and processor 14, and obtain immediate response in the terms of advice as to whether that food product should be consumed or that activity commenced.

As previously explained, the system also maintains an update of information, including calories consumed, exercise undertaken, stress and sleep levels so that an up to date database of the person's well being and characteristics are maintained. This enables this system to prompt the user should sleep patterns, stress, or the like, fall outside the parameters which may be regarded as healthy, having regard to the user's lifestyle and general health and wellbeing.

The microprocessor 14 then ascertains the food product concerned and from the databank stored in memory 12b retrieves the characteristics relating to the food product and supplies those characteristics to memory 24 which maintains a count or calculation of the amount of that particular characteristic which has been consumed by a user in a given period such as a day.

Depending on the food product concerned, the microprocessor 14 may prompt the user to input additional information. For example, the product may come in different sizes or the consumer may only be proposing to consume part of the product and can therefore prompt the user to input information relating to size, on the keypad 14 or the amount of the product which the consumer intends to consume such as ¼,½ or ¾, etc. The processor 14 then makes the appropriate calculation of the characteristics such as calories, fat, etc. which the consumer will consume when the consumer eats that product or part of the product and the number of calories, etc. is supplied to the memory 24 which maintains an updated count of the calories, fat, etc. consumed by the user in a given period. If the amount of a particular characteristic consumed by a user approaches or reaches a pre-determined level which may be stored in memory 12a or in one of the other memories, then the microprocessor can transmit information back to the user telephone 15 for display indicating that the user has consumed or is close to consuming his or her daily requirement of that particular characteristic. The particular daily requirement may be dependent on the information stored in the memory 12a which contains the user data or maybe simply a recommended daily allowance of a particular characteristic stored in the memory 12b.

In one embodiment of the invention the device preferably provides a warning when the user inserts data relating to a food product to be consumed, if the consumption would take the user beyond the daily requirement or to a particular level approaching the daily requirement, or indicative of the fact that the user is consuming too much of the particular substance within a given period in a day. This enables the user to be alerted before the user consumes any product so the user can make an informed decision as to whether the user does in fact wish to consume the product.

As well as displaying the information on the user telephone 15, the microprocessor 14 may trigger an audible alarm on the telephone 15, prompting the user to observe the display to obtain an indication that the daily intake of a particular characteristic has been reached or is approaching a particular level. The display or audible alarm may be triggered at any particular time such as if the user consumes a significant quantity of a particular characteristic earlier than a given period so as to alert the user that, for example, by 10 am in a day the user may have already consumed ¾ of his or her daily calorie requirement so the user is alerted as quickly as possible to the fact that the user should be careful concerning further intake of calories during the remaining course of the day so as not to exceed his or her required level. The audible alarm may be given by simply making a normal telephone call to the mobile phone 15, which causes the telephone to ring so that the telephone is answered by the user, and the message can be provided as a voice message or simply an indication to view the telephone for an SMS message.

If the food product concerned is not provided with a barcode such as a fruit item, for example an apple, the user can input information into the keypad of the telephone 15 by pressing a menu button for example which may display menu items such as fruit, vegetable, etc., typical of products which do not normally carry barcodes and then by scrolling through that memory item, select the item in question so that the data relating to that item is transmitted to processor 14 so the relevant characteristics can be extracted from the memory 12b and stored in the memory 24 so the running count of the characteristics consumed by the user over the course of the day can be maintained and monitored.

Furtherstill, in relation to food which is consumed at home, a menu of food items may be provided at the home with appropriate barcodes which can be scanned when the user intends to consume a particular item. This aspect of the preferred embodiment is advantageous for home cooked meals or the like which obviously do not come with a barcode or any other identifying data. A menu of food products and ingredients in food products which are typically used in the home can be kept at the home with a barcode which provides data relating to the calorie content, fat content, etc, and also another barcode for providing information relating to the amount of the product which will be added into a meal or consumed by the user.

In basic versions of the system of this embodiment the menu may simply include standard meals such as spaghetti bolognaise, steak and vegetables, roast chicken, soup, etc. However, in more sophisticated or complex versions of the invention, rather than merely listing the entire meal, all ingredients which typically make up a meal can be listed so the user can scan each of those ingredients which will actually be included in the meal to obtain a more accurate indication of the actual calorie content, fat content, etc. of the meal which is to be consumed.

The embodiment of FIGS. 1 and 2 may also include supplementary input for inputting data relating to the calories burnt by a user during the course of the user's daily activity. The ancillary input 25 may be in the form of a heart monitor which monitors heart rate so that an indication of the calories burnt by a user during normal daily activity, sport or other activities can be inputted into the processor 14 via mobile telephone 15. This enables an indication of calories burnt to be subtracted from calories consumed to provide a better indication of net calories required by a user during the course of a day or, for that matter, net calories which are lost. The calorie count acquired by the apparatus can then be discounted in accordance with calories burnt depending on the needs of the user and the objectives of the user in monitoring intake of the various food characteristics.

FIG. 4 illustrates an embodiment of the invention which has particular application in restaurants or other environments where food is served in a ready to eat form. In this embodiment, menus in the restaurants can include a barcode 80 alongside the item name illustrated by reference 82 which provides information relating to the calorie content, fat content, etc. of the menu item. If a user wishes to ascertain the characteristics of the menu item, the user can scan the barcode 80 with the scanner of the mobile telephone 15 for transmission to processor 14.

The preferred embodiments of the present invention have the advantage that they not only monitor the calorie intake, fat intake, etc. of a consumer during the course of a day or other given period, but also enable this to be done without the user needing to know or find the calorie content, fat content or the like of the product. That information is stored in the databank which the user accesses by simply scanning the product or inserting data relating to the product such as a product name. The preferred embodiments also have the advantage of alerting the user when daily intake approaches a pre-determined limit by either the display or audible alarm so the user is appraised of the fact that he or she is consuming too much of a particular item in a given period.

The preferred embodiments of the invention have particular application to people who may, for medical reasons or otherwise, have strict dietary requirements and need to carefully monitor intake of particular characteristics of food products. The invention also has obvious application for all people including those who wish to merely maintain an indication of their consumption for their own well-being.

Since modifications within the spirit and scope of the invention may readily be effected by persons skilled within the art, it is to be understood that this invention is not limited to the particular embodiment described by way of example hereinabove.

1. A health and wellbeing advisory system, including:
a central database which holds information and data relating to a specific user, and information relating to health and wellbeing parameters, the central database being contactable by a transmitter used by a user, which transmitter is able to transmit information relating to a user to the database;
processing means for receiving the user information and determining a response to the user information based on the data relating to the specific user and/or the information relating to the health and wellbeing parameters; and
return transmitting means for transmitting a response back to the user's transmitter.

2. The system of claim 1 wherein the processing means is stored with predetermined protocols against which the response is determined based on those protocols, as well as the user information transmitted to the central database, and the information and data relating to the specific user and/or the health and wellbeing parameters.

3. The system of claim 1 wherein the transmitter is a wireless transmitter.

4. The system of claim 1 wherein the wireless transmitter is a mobile telephone and the database receives information identifying the user by the mobile telephone number which is transmitted with the data to the database, and the return transmitting means is a mobile telephone transmitting means which transmits the response to a user by way of a mobile telephone call.

5. The system of claim 4 wherein the information is transmitted from the user mobile telephone by an SMS message, and the response is transmitted back to the user from the return transmitting means by an SMS message.

6. The system of claim 1 wherein the database includes a first database section which includes personal records at which specific user information is stored relating to the specific users of the system, and a second database section in which health and wellbeing parameters are stored.

7. The system of claim 5 wherein the database includes an SMS modem for receiving the information from the user and for supplying the information to the database and the processing means.

8. The system of claim 1 wherein a health and nutrition evaluation centre is coupled to the system so that information relating to a specific user can be input into the system by consultation with a lifestyle consultant.

9. The system of claim 8 wherein the health and nutrition evaluation centre is connectable to the system over the Internet.

10. The system of claim 1 wherein the user transmitter includes input means for inputting data into the system for transmission to the database.

11. The system of claim 10 wherein the input means comprises a scanner connectable to the wireless transmitter so that a bar code of a product can be scanned to provide the information relating to the product.

12. The system of claim 10 wherein the transmitter includes a menu of products and the input is by selection from a draw down menu in the transmitter.

13. The system of claim 10 wherein the transmitter includes voice recognition software and the input is by way of a spoken word which is recognised by the software in the transmitter, so that data relating to the voice input is transmitted by the transmitter to the database.

14. The system of claim 1 wherein the user transmitter includes a display for displaying the quantity of the food characteristic consumed by a user.

15. The system of claim 1 wherein the processing means also includes processing means for calculating an ideal intake of calories having regard to the user information and data and the requirements or end result the user wishes to achieve.

16. The system of claim 1 wherein the system also allows input of information relating to the calories burnt by a user during the user's daily activity.

17. A diet monitoring system, including:
a mobile telephone for communicating with a central facility to provide a communication signal, and having input means for input of information relating to a food product to be consumed; and
a central facility having:
(i) memory means for storing a databank of data relating to food products and characteristics of those food products and information and data relating to a specific user;
(ii) receiving means for receiving the communication signal;
(iii)processing means for receiving the communication signal and processing the signal so that the characteristic of a food product to be consumed by the specific user can be determined from the memory means and stored to provide a measure of the consumption of the particular characteristic in a given time period; and
(iv) transmitter means for transmitting information relating to the food to be consumed back to the mobile telephone.

18. The system of claim 17 wherein the mobile telephone includes indicating means for providing an indication as to the quantity of a food characteristic consumed by the user.

19. The system of claim 17 wherein the indicating means comprises a display for displaying the quantity of total characteristic consumed by a user.

20. The system of claim 17 wherein the mobile telephone also include alarm means for generating an alarm when the total amount of a particular food characteristic consumed by a user approaches or reaches a pre-determined level.

21. The system of claim 17 wherein the input means comprises a scanner so that a barcode on a food product can be scanned to input data relating to the food product such as calorie content, fat content, fibre content, alcohol content, etc.

22. The system of claim 21 wherein the input means further includes a keypad for manual input of data in the event that a food product does not include a barcode.

23. A diet monitoring system including:
a mobile telephone for transmitting information and receiving a response;
memory means for storing data relating to a plurality of food products and characteristics of those food products;
input means associated with the telephone for receiving data relating to a food to be consumed by a user and transmitting that data by a mobile telephone call;
processing means for comparing the data received by the input means with the data stored in the memory so as to extract from the memory an amount of at least one of the characteristics of the food the consumer is to consume, and transmitting the response to the mobile telephone; and
display means associated with the telephone for displaying that response.

24. The system of claim 23 wherein the system also includes alarm means for generating an alarm when the total amount of a particular food characteristic consumed by a user approaches or reaches a pre-determined level.

25. The system of claim 23 wherein the input means comprises a scanner so that a barcode on a food product can be scanned to input data relating to the food product such as calorie content, fat content, fibre content, alcohol content, etc.

26. The system of claim 23 wherein the input means further includes a keypad for manual input of data in the event that a food product does not include a barcode.
